(19) United States
(12) Patent Application Publication
(10) Pub. No.: US 2003/0236207 A1
(43) Pub. Date: Dec. 25, 2003

(54) Particulate complex for administering nucleic acid into a cell

(75) Inventors: Arnaud Debin, Pompertuzat (FR); Roger Kravtzoff, St. Felix de Lauragais (FR); Marinette Moynier, Ramonville Saint Agne (FR); Ignacio De Miguel, Plaisance du Touch (FR); Olivier Balland, Pechabou (FR); Philippe Pajot, Toulouse (FR); Jocelyn Vaz Santiago, I'Union (FR); Paul von Hoegen, Ohain/Lasne (BE)

Correspondence Address: SCHNADER HARRISON SEGAL & LEWIS, LLP, 1600 MARKET STREET, SUITE 3600, PHILADELPHIA, PA, 19103

(73) Assignee: Biovector Therapeutics, S.A., Labege Cedex (FR)

(21) Appl. No.: 10/280,408

(22) Filed: Oct. 25, 2002

Related U.S. Application Data
(63) Continuation of application No. PCT/IB01/00873, filed on Apr. 24, 2001.

(30) Foreign Application Priority Data
Apr. 25, 2000 (US) 09/557717
Dec. 22, 2000 (US) 09/745644

(51) Int. Cl. A61K 48/00; C07H 21/04; A61K 31/715

(52) U.S. Cl. 514/44; 514/54; 536/23.2; 536/18.7

(57) ABSTRACT

A particulate complex including a nucleic acid and a biodegradable cationized polyhydroxylated molecule, wherein said molecule has a charge up to approximately 1.0 meq/g.

### BACKGROUND OF INVENTION

This invention concerns particulate complexes and their use for administering a nucleic acid molecule into a cell.

Many systems for administering active substances into cells are already known, such as liposomes, nanoparticles, polymer particles, immuno- and ligand-complexes and cyclodextrins (Drug Transport in antimicrobial and anticancer chemotherapy. G. Papadakou Ed., CRC Press, 1995). However, none of these systems has proved to be truly satisfactory for the in vivo transport of nucleic acids such as, for example, deoxyribonucleic acid (DNA).

Satisfactory in vivo transport of nucleic acids into cells is necessary for example, in gene therapy. Gene therapy is the transfection of a nucleic acid-based product, such as a gene, into the cells of an organism. The gene is expressed in the cells after it has been introduced into the organism.

Several methods of cell transfection exist at present. These methods can be grouped as follows:

use of calcium phosphate, microinjection, protoplasmic fusion;

electroporation and injection of free DNA.

viral infection;

synthetic vectors.

Methods in the first group are not applicable to in vivo transfection. As a result, most initial clinical trials of gene therapy taking place today are based upon the utilization of retroviral or adenoviral vectors. Examples of viral vectors that have been tried include retroviral, herpes virus, and adenoviral vectors. These retroviral vectors can be effective in stably transfecting heterologous genes into some cells for expression. However, clinical utilition of vectors of viral origin appears problematic because of their specificity, immunogenicity, high production costs, and potential toxicity.

Electroporation and injection of free DNA offer a useful alternative. These methods are, however, relatively ineffective, and limited to local administration only.

There is increasing interest in the use of synthetic vectors, such as lipid or polypeptide vectors. Synthetic vectors appear to be less toxic than the viral vectors.

Among synthetic vectors, lipid vectors, such as liposomes, appear to have the advantage over polypeptide vectors of being potentially less immunogenic and, for the time being, more efficient. However, the use of conventional liposomes for DNA delivery is very limited because of the low encapsulation rate and their inability to compact large molecules, such as nucleic acids.

The formation of DNA complexes with cationic lipids has been studied by various laboratories (Felgner et al., PNAS 84, 7413-7417 (1987); Gao et al., Biochem. Biophys. Res. Commun. 179, 280-285, (1991); Behr, Bioconj. Chem. 5, 382-389 (1994)). These DNA-cationic lipid complexes have also been designated in the past using the term lipoplexes (P. Felgaer et al., Hum. Genet. Ther., 8, 511-512, 1997). Cationic lipids enable the formation of relatively stable electrostatic complexes with DNA, which is a poylanionic substance.

The use of cationic lipids has been shown to be effective in the transport of DNA in cell culture. However, the in vivo application of these complexes for gene transfer, particularly after systemic administration, is poorly documented (Zhu et al., Science 261, 209-211 (1993); Thierry et al., PNAS 92, 9742-9746 (1995); Hofland et al., PNAS 93, 7305-7309 (1996)).

Cationized polymers have also been investigated as vector complexes for transfecting DNA. For example, vectors called “Neutraplexes” containing a cationic polysaccaride matrix have been described in U.S. Pat. No. 6,096,335 owned by Biovector Therapeutics of Toulouse, France. Such vectors also contain an amphiphilic compound, such as a lipid.

Chitosan conjugates having pendant galactose residues have also been investigated as a gene delivery vector. See Murata et al., “Possibility of Application of Quaternary Chitosan Having Pendant Galactose Residues as Gene Delivery Tool,” Carbohydrate Polymers, 29(1):69-74 (1996); Murata et al., “Design of Quaternary Chitosan Conjugate Having Antennary Galactose Residues as a Gene Delivery Tool,” Carbohyarate Polymers 32:105-109 (1997). Chitosan is cationic natural polysaccharide. However, chitosan is strongly charged. Therefore, chitosan will complex too strongly to the nucleic acid to permit the proper release of the nucleic acid when reaching the target cells.

Galactosylated polyethyleneimine/DNA complexes have also been investigated See Bettinger, et. al., “Size Reduction of Galactosylated PEI/DNA Complexes Improves Lectin-Mediated Gene Transfer into Hepatocytes,” Bioconjugate Chem., 10:558-561 (1999). However, such complexes rely upon a decrease in pH in lysosomes in order to release the DNA. Therefore, the mechanism cannot be extended to in vivo applications.

Therefore, there is a need for an improved particulate vector for administering a nucleic acid molecule into a cell.

### SUMMARY OF INVENTION

These and other inventions, as will be apparent to those having ordinary skill in the art, have been achieved by providing a particulate complex comprising a nucleic acid and a biodegradable cationized polyhydroxylated molecule, wherein the molecule has a charge up to approximately 1.0 meq/g.

### BRIEF DESCRIPTION OF DRAWINGS

FIG. 1 is a graph demonstrating β-galactosidase expression in muscle with and without use of the complexes of the invention.

FIG. 2 is a graph demonstrating production of antibodies against beta-galactosidase after intramuscular administration of DNA/glucidex6-GTMA.

FIG. 3 is a graph demonstrating induction of cellular response (elispot gamma-IFN) after intramuscular administration of DNA/glucidex6-GTMA.

FIG. 4 is a graph demonstrating induction of CTL response after immunization with DNA/glucidex6-GTMA.

### DETAILED DESCRIPTION OF THE INVENTION

The inventors have surprisingly discovered that a particulate complex between a nucleic acid molecule and a biodegradable cationized polyhydroxylated molecule provide advantages for transfecting a nucleic acid molecule into a cell. The charge on the vector should be sufficient to stably bind the nucleic acid. At the same time, the charge should remain low enough to allow for the necessary release of the nucleic acid molecule.

“Nucleic acid” is defined as any single or double-stranded polynucleotide. Nucleic acids include, for example, double or single stranded DNA, RNA or a mixture thereof. The nucleic acid can include natural or chemically modified sequences, or derivatives thereof. The nucleic acid can also be a mixture of different nucleic acids.

The polynucleotide can be any size, depending on its purpose. The term “polynucleotide” as used herein, includes RNA or DNA sequences of more than one nucleotide in either single chain, duplex or multiple chain form. The polynucleotide may, for example, be an oligonucleotide. An oligonucleotide is a short length of single stranded polynucleotide chain, usually less than 30 bases long. The polynucleotide typically contains more than thirty bases and can also be much longer, with no upper limit.

The polynucleotide preferably includes the structural (coding) region of a gene. The polynucleotide may also encode signal sequences, such as promoter regions, operator regions, translocation signals, termination regions, combinations thereof or any other genetically relevant material. The gene being transfected can include only the structural region, and rely upon the non-structural regions (e.g. signal sequences) existing in the DNA of the cell being transfected. The polynucleotide can also encode only a signal sequence, if desirable. Examples of oligonucleotides which can be transfected are antisense oligonucleotides (DNA and RNA), ribozymes, and triplex-forming oligonucleotides. Optionally, the nucleic acid can be naked or can be part of a vector, other than the particulate complex of the invention (e.g. plasmid DNA).

The nucleic acid is complexed to a cationized polyhydroxylated molecule. Preferred polyhydroxylated molecules include, for example, saccharides, polyglycols, polyvinyl alcohol, polynoxylin. Saccharides include monosaccharides, oligosaccharides, and polysaccharides. The saccharides can be natural or synthetic.

Examples of polysaccharides include, starch, glycogen, amylose, and amylopectin. Examples of oligosaccharides include maltose, maltodextrin, lactose, and sucrose. Examples of monosaccharides include, galactose, mannose, fucose, ribose, arabinose, xylose, and rhamnose.

Glucidex is an example of a maltodextrin that can be used in the complex of size of the molecule. For example, as shown in Table I in Example 1, Glucidex 2 has an average molecular weight of 10 kDa; Glucidex 6, made up of sixteen sugar units, has an average molecular weight of 3 kDa; Glucidex 12 has an average molecular weight of 1.4 kDa, and Glucidex 21 has an average molecular weight of 0.8 kDa.

The polyhydroxylated molecule can be cationized by grafting thereto a suitable cationic moiety. Examples of such cationic moieties include secondary or tertiary amino groups, quaternary ammonium ions, or a combination thereof. Glycidyl trimethylammonium (GTMA) is a preferred cationic group.

The cationized polyhydroxylated molecule is biodegradable. “Biodegradable” means that the molecule is able to be degraded by a hydrolytic enzyme naturally present in mammals in order to obtain fragments which are metabolized and/or eliminated from the body. Examples of such enzymes include glycosidases, amylase, and glucosaminidase.

The cationized polyhydroxylated molecule has a positive charge up to approximately 1.0 meq/g. The charge may be as low as 0.001 meq/g, preferably 0.01 meq/g, and more preferably 0.1-0.5 meq/g. Molecules which have a charge greater than 1.0 meq/g are less biodegradable. Therefore, polyhydroxylated molecules do not include chitosan, quaternarized chitosan, or DEAE-Dextran.

The optimal charge on the polyhydroxylated molecule will vary according to the size of the molecule and the nature of the nucleic acid to be grafted. The optimal charge can be determined by one of ordinary skill in the art. It is preferred that the polyhydroxylated molecule has a charge between approximately 0.1 and approximately 0.85 meq/g. In the case of GTMA and Glucidex, such charge expressed in meq/g corresponds to 1 to 10 moles of GTMA grafted per mole of Glucidex 2, or 0.3 to 3 moles of GTMA grafted per mole of Glucidex 6.

It is preferred that the cationized polyhydroxylated molecule have a molecular weight of between about 0.18 KDa and 1,000 KDa, more preferably between approximately 0.5 KDa and approximately 500 KDa.

The cationized polyhydroxylated molecule and nucleic acid are combined to form the particulate complex of the invention. The polyhydroxylated molecule and nucleic acid can be combined or grafted by methods known in the art. Because of their opposite charges, the polyhydroxylated molecule and the nucleic acid can be combined, for example, by simply mixing them in a solution. The order of mixing is not critical. For instance, saccharide powder can be solubilized in a saccharide solution. Additional steps can be used in the process, e.g. homogenization, lyophilization, concentration, evaporation, and ultrafiltration.

The particulate complex optionally includes a lipid component. In a preferred embodiment, the particulate complex lacks a cationic lipid component.

The particulate complex can include nucleic acids and biodegradable cationized polyhydroxylated molecules of various sizes. Therefore, the molecular weight of the particulate complexes of the invention will vary. The preferred size of the particulate complex as a whole is between approximately 100 nm to approximately 10 μm, more preferably between 200 nm and 1 μm.

The global charge of the particulate complexes of the invention is the result of the relative number of positive to negative charges, and can be described in terms of charge ratio. In this specification, a charge ratio is defined in accordance with Felgner, et al. “Nomenclature for Synthetic Gene Delivery Systems,” Human Gene Therapy, 8:511-512(1997):

Charge ratio=Positive charge of polyhydroxylated molecule in meq/g×Mass (g) Negative charge of nucleic acid in meq/g×Mass (g)

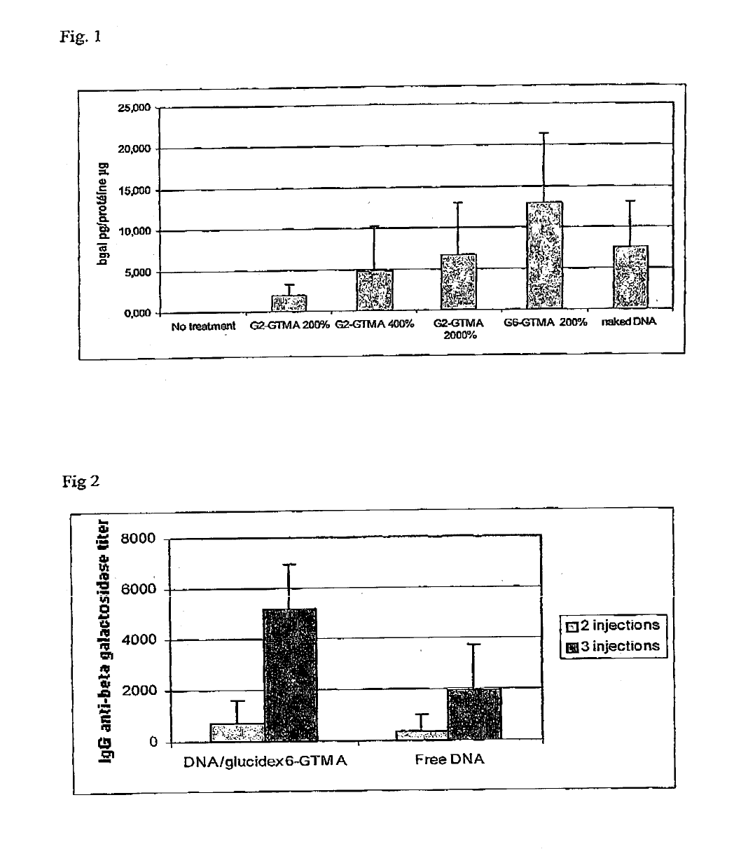

The positive charge of the polyhydroxylated molecule includes any cationic constituents. The negative charge of the nucleic acid includes any anionic constituents. The charge ratio can also be expressed in terms of a percentage by multiplying the resulting fraction by 100. The charge ratio is expressed in this manner in FIG. 1.

The zeta potential of a solution comprising the particulate complexes is an experimental parameter that is directly correlated to the cationized polyhydroxylated molecule/nucleic acid charge ratio. When the charge ratio is <1, the zeta potential is negative, which indicates a negatively charged surface on the particles. Alternatively, when this charge ratio is >1, the zeta potential is positive, which indicates a positively charged surface on the particles. Experimentally, the zeta potential, expressed as mV, is indicative of the particle charge ratio. The zeta potential can be determined by a zeta potential analyzer.

The particulate complexes of the present invention may be positive or negative. The choice of a positive or negative complex is guided by the route of administration. In case of intravenous administration, a negative complex is more appropriate. For mucosal administration, a positive complex is preferred.

In a preferred embodiment the complex has a charge ratio of cationized polyhydroxylated molecule to nucleic acid between approximately 0.3 to 1, wherein the complex is globally negative. In another preferred embodiment, the complex has a charge ratio of cationized polyhydroxylated molecule to nucleic acid between 1 to approximately 20, wherein the complex is globally positive.

It has been discovered that there is a close relationship between the charge ratio of the particulate complex, and the kinetics of release of the nucleic acid. The kinetics of the release of the nucleic acid, in turn, affects the efficacy of transfection of the released nucleic acid. The optimal charge ratio for each complex can be determined by a person of ordinary skill in the art, within the parameters set forth above.

When the complex is globally positive, there is more cationized polyhydroxylated molecule than is necessary to fully complex with the nucleic acid.

In a separate preferred embodiment, a solution is provided that includes a globally positive complex as described above and further includes excess polyhydroxylated molecule not complexed to nucleic acid. Without being bound by theory, it is believed that the excess polyhydroxylated molecule, such as a polysaccharide, may act as an enhancer for transfection. This may be due to the interaction of the polyhydroxylated molecule or its degradation products with DNA and with the cellular membranes, enhancing the penetration of DNA into the cells.

A method is also provided for protecting a nucleic acid molecule when transfecting the nucleic molecule into a cell. The method includes complexing the nucleic acid with a cationized polyhydroxylated molecule to form a particulate complex as described above.

In another separate embodiment, a method is provided for administrating a nucleic acid molecule into a cell. The administration into the cell can occur ex vivo or to a mammalian cell in vivo. The method includes complexing the nucleic acid with a cationized polyhydroxylated molecule to form a particulate complex as described above. The particulate complex is then utilized in transfecting the nucleic acid molecule into a cell by known means.

In one embodiment, the nucleic acid molecule encodes a peptide or protein that shares at least one epitope with an immunogenic protein found on a pathogen. The pathogen may be, for example, a virus, bacteria, or protozoa. Examples of viral pathogens include human immunodeficiency virus, HIV; human T cell leukemia virus, HTLV; influenza virus; hepatitis A virus, HAV; hepatitis B virus, HBV; hepatitis C virus, HCV; human papilloma virus, HPV; Herpes simplex 1 virus, HSV1; Herpes simplex 2 virus, HSV2; Cytomegalovirus, CMV; Epstein-Barr virus, EBV; rhinovirus; and, coronavirus. Examples of bacteria include meningococcus, tuberculosis, streptococcus, and tetanus. Examples of protozoa include malaria or Trypanosoma. The complex is administered to the mammal so as to induce an immune response.

The method is also used for non-pathogen mediated mammalian pathologies where modulation of the immune response is important. Some examples of non-pathogen mediated pathologies include cancer, autoimmune disease, and allergies.

The particulate complex may be administered to the mammal by any known means. For example, methods of administration can include mucosal, intratumoral, pulmonary, intravenous, intramuscular, intraparietal, intraoccular, cutaneous, intradermal, subcutaneous, or a combination thereof.

The mammal treated in accordance with the method of the invention may be any mammal, such as farm animals, pet animals, laboratory animals, and primates, including humans. Farm animals include, for example, cows, goats, sheep, pigs, and horses. Pet animals include, for example, dogs and cats. Laboratory animals include, for example, rabbits, mice, and rats.

In another embodiment, the nucleic acid comprises at least the coding region of a therapeutic protein in order to synthesize the therapeutic protein in the cell. Some examples of therapeutic proteins include enzymes, hormones, antigens, clotting factors, regulatory proteins, transcription factors and receptors. Some specific examples of therapeutic proteins include erythropoietin, somatostatin, tissue plaminogen activator, factor VIII, etc. The nucleic acids could be designed to obtain an intracellular oligonucleotide, such as ribozymes, antisense, and gene transcripts. In this embodiment, the nucleic acid comprises at least the coding region of an oligonucleotide used to inhibit expression of a gene.

In a separate embodiment, the particle complex is administered in a pharmaceutical composition. The pharmaceutical composition may be manufactured by known means and can include typical ingredients. For example, the pharmaceutical composition can include a pharmaceutically acceptable diluent or carrier, a buffer, a preserving or stabilising agent, an adjuvant, and/or an excipient.

In a preferred embodiment, the pharmaceutical composition further includes a transfection enhancer. Examples of transfection enhancers include lipids, detergents, enzymes, peptides, or enzyme inhibitors.

### EXAMPLES

### Example 1

Preparation of Biodegradable Cationized Saccharides having a Charge between 0.2 and 1 mEq/g

Twenty grams of maltodextrins of various molecular weight (Glucidex 2, Glucidex 6, Glucidex 12, Glucidex 21, Roquette, Lille, France) or amylopectin (Waxilys 200, Roquette) were dispersed in 2 N NaOH as indicated in Table I. When the suspension was homogeneous, glycidyl trimethylammonium (GTMA) chloride (Fluka, Saint Quentin Fallavier, France) was added. The degree of ionic grafting on the saccharide was adjusted by varying amount of glycidyl trimethyl ammonium chloride (Table I). This reaction lead to grafting of 3-(N, N, N trimethylamino)-2-ol-1-propyloxy groups on the sugars.

The reaction mixture was stirred for 5 hours at room temperature. The solution of grafted saccharides was then brought to pH between 5 and 7 with concentrated acetic acid and then dispersed by addition of distilled water.

To remove all the salts and reaction by-products, the suspension was ultrafiltered (tangential ultrafiltration on Minisette system, Filtron, Pall Gelman Sciences) with a membrane having an appropriate cutoff according to the molecular weight of the polymer (see Table I). Smaller molecular weight (Glucidex 12 and Glucidex 21) polymers were precipitated by absolute ethanol.

The suspension polymers were sterilizated by filtration through 0.2 μm polyethersulfone membrane (SpiralCap® capsule, Pall Gelman Sciences). The grafting yield was determined by nitrogen elemental analysis by proton NMR. The results are presented in table I.

TABLE 1

| | | Average | | | |
|---|---|---|---|---|---|
| | 2N | molecular | | Molecular | |
| | NaOH | weight | GTMA | cutoff | Charge |
| Saccharide | (ml) | (daltons) | chloride (g) | (daltons) | (mEq/g) |
| Glucidex 2 | 40 | 10,000 | 1.44 | 30.000 | 0.30 |
| | 40 | 10,000 | 2.34 | 30.000 | 0.46 |
| Glucidex 6 | 40 | 3,000 | 1.44 | 3.000 | 0.30 |
| 40 | 3,000 | 2.34 | 3.000 | 0.45 | |
| 40 | 3,000 | 4.68 | 3.000 | 0.85 | |
| Glucidex 12 | 30 | 1,400 | 1.70 | — | 0.32 |
| 30 | 1,400 | 3.74 | — | 0.60 | |
| Glucidex 21 | 20 | 800 | 4.68 | — | 0.76 |
| Waxilys 200 | 60 | 800,000 | 3.12 | 100.000 | 0.54 |

### Example 2

Preparation of DNA/biodegradable Cationized Saccharide Complexes

DNA/biodegradable cationized saccharide complexes were formed by mixing a solution containing 100 μg DNA with the required quantity of cationized saccharides in a final volume of 1 ml under vortex stirring. The quantity of added cationized saccharides was dependent on the required DNA/polymer ratio. After 30 min. incubation at room temperature, 1 ml of complex solution was mixed with 125 μl acetate buffer 200 mM pH 5.3. The resulting mixture was homogeneized with a vortex mixer and stored at 4° C.

Characteristics of the DNA/biodegradable Cationized Saccharides Complexes.

The visual appearance of the complexes was clear and homogeneous. Their characteristics are summarized in Table II. DNA/biodegradable cationized saccharide complexes appeared to range from 60 to 3,000 nm in diameter as determined by light scattering measurement (Coulter N4 SD).

TABLE II

| | | | Charge | | |
|---|---|---|---|---|---|
| | | | ratio | | Zeta |
| | | Charge | Polymers/ | | potential |
| DNA | Polymers | mEq/g | DNA | Size | mV |
| 100 μg | Glucidex 6 | 0.45 | 2 | 74 nm | +25 |
| 100 μg | Glucidex 6 | 0.45 | 4 | 70 nm | +40 |
| 100 μg | Glucidex 6 | 0.85 | 4 | 90 nm | +30 |
| 100 μg | Glucidex 2 | 0.46 | 0.5 | 180 nm | −20 |
| 100 μg | Glucidex 2 | 0.46 | 2 | 120 nm | +22 |
| 100 μg | Glucidex 2 | 0.46 | 4 | 115 nm | +28 |
| 100 μg | Glucidex 2 | 0.50 | 20 | 70 nm | +26 |
| 100 μg | Glucidex 2 | 0.3 | 4 | 120 nm | +18 |
| 100 μg | Waxilys | 0.54 | 0.5 | 0.5-2 μm | ND^{a} |
| 100 μg | Waxilys | 0.54 | 2 | 1-2 μm | ND |

^{a}ND: not determined

The percentage of DNA association was estimated by 1% agarose gel, TAE 1×. 20 μl of the formulation were mixed with 2 μl of loading solution 10×(glycerol 50%, bromophenol blue 0.025%), then 20 μl of the resulting solution were loaded per well. The calculated quantity of DNA loaded was 1.6 μg/well. As a control, the same quantity of DNA has been loaded. After 40 min migration of the gel at 90 V, the gel was stained in a BET bath before visualization under U.V. light.

No migration of DNA was detected for the loaded DNA/cationized saccharide complexes tested. Migration was only observed for the free DNA not grafted to a cationized saccharide. These results demonstrate that 100% of the initial DNA input dose was complexed by the saccharide.

### Example 3

Biodegradability of the Cationized Saccharides, and Liberation of the Entrapped DNA

The biodegradability of the DNA/cationized saccharides complexes was assayed by an in vitro degradation assay. 200 μl of formulations were added to 40 μl of amylase cocktail (1 mg/ml α-amylase, 1 mg/ml amyloglucosidase in citrate buffer 100 mM pH5). After overnight incubation under rotative agitation at room temperature, 20 μl of the treated samples were loaded on 1% agarose gel.

When the amylase was omitted, no migration of DNA was detected for the loaded DNA/cationized saccharide complexes. When the amylase was added, a significant part of the DNA migrated inside the gel. For the complexes having a low saccharide/DNA ratio, all the DNA was recovered and migrated at the same position as free DNA.

These results demonstrate that the polymer is biodegradable, which permits DNA release. Moreover, after release, no modification of DNA could be detected. As an example, no change of supercoiled/relaxed ratio is detected, which indicates that no nicking of DNA occurs during the formation of the particles.

### Example 4

In Vivo Transfection Studies

I Materials and Methods

Plasmid DNA

Gene transfer studies were carried out with pCMVβ plasmid DNA (Clontech) coding for β galactosidase. The plasmid DNA was purified by double chloride cesium gradient centrifugation (BioServe Biotechnologies, Ltd, USA) and resuspended in purified water. The concentration of DNA was 4.7 mg/ml as calculated based on absorbance of ultraviolet light (OD 260). Endotoxine level was 2.5 IU/mg as determined by the Limulus assay (Charles River, France). DNA solutions were stored at −20° C. until required for use. DNA was administered either as pure plasmid DNA on saline (naked DNA) or formulated with the biodegradable cationized saccharides.

DNA/Biodegradable Cationized Saccharide Complexes

The biodegradable cationized saccharide was synthesized as described above in Example 1. The DNA/glu2 and DNA/glu6 complexes were prepared as described above in Example 2.

In Vivo Gene Transfer

Animals.

All experiments were carried out using 8-9 week-old female BALB/c mice (Janvier, France) with 4 mice per experimental or control group.

Intramuscular Administration.

Each animal received one intramuscular injection of 8 μg of naked or formulated DNA in a total volume of 100 μl in each quadriceps. The injections was made using a 27×½ gauge needle fitted with a polyethylene tubing which limited the penetration to 2 mm.

Evaluation of Reporter Gene Expression.

The entire quadriceps muscle was collected from each mouse leg at day 7 postinjection. Muscles were frozen in liquid nitrogen immediately after collection and stored in 2.0 ml Eppendorf tubes at −80° C. Frozen muscles were individually pulverized into a fine powder by hand grinding with a dry ice-chilled porcelain mortar and pestle and the powder was stored in the same tube at −80° C. until extraction. One ml of β-galtosidase lysis buffer (100 mM potassium phosphate pH 7.8, 0.2% Triton X-100, 1 mM DTT, 0.2 mM phenylmethylsulfonyl fluoride and 5 μg/ml leupeptin) was added. The latter three components were added just before use. The samples were vortexed for 15 min, frozen and thawed three times using alternating liquid nitrogen and 37° C. water baths, and centrifuged for 5 min at 13.000 RPM. The supernatant was transferred to another 1.5 ml eppendorf tube and stored at −80° C. until tested for β galactosidase enzyme assays.

β galactosidase enzyme assays using MUG (Sigma, France) as a β-galactosidase substrate were performed in a reaction buffer containing 25 mM Tris-HCl (pH 7.5); 125 mM NaCl; 1 mM DTT; and 2 mM MgCl₂. Just before use, MUG substrate (prepared as a 20 mg/ml slurry in ethanol) was added to a find concentration of 100 μg/ml.

Standards were prepared by adding known quantities of purified β-galactosidase (Promega) in 50 μl of control muscles extract supernatant (over the range of 200 pg to 200 ng in 50 μl). Samples were assayed by addition of 200 μl of complete reaction buffer to 50 μl of sample in a 1.5 ml eppendorf tube and incubated at 37° C. for 1 hour. The reactions were stopped by adding 50 μl of cold 25% trichloroacetic acid, chilled on ice for 5 min and clarified by centrifugation for 2 min at room temperature. 200 μl aliquots of each sample were added to 2 ml of glycine/carbonate buffer, vortexed, and read in a spectrofluorimeter using 366 nm excitation and 442 nm emission.

Protein concentrations of muscle extracts were determined using the microBCA assay (Pierce). β galactosidase enzyme concentration present in the sample was measured and expressed as ng β galactosidase/mg of total protein after normalization with β galactosidase standard curve and protein concentrations.

II Results

The results are shown in FIG. 1. DNA formulated with cationic Glucidex 2 and Glucidex 6 and administrated intramuscularly allows high levels of β galactosidase expression in muscle. The highest expression was obtained with DNA/glu2 at the charge ratio of 20 and DNA/glu6 at the charge ratio of 2. Also, an increased amount of expression was observed when the charge ratio was progressively increased for glu2. Most importantly, the amount of expression with DNA/glu6 at the charge ratio of 2 was higher than with naked DNA.

### Example 5

Immunological Study

I Materials and Methods

Plasmid DNA

Immunization studies were carried out with pCMVβ plasmid DNA (Clontech) coding for β galactosidase described in Example 4.

DNA/Biodegradable Cationized Saccharide Formulations.

The biodegradable cationized saccharide was synthesized as described above in Example 1. The DNA/Glucidex G2-GTMA and DNA/Glucidex G6-GTMA formulations were prepared as described above in Example 2.

DNA Immunization

Animals.

Immunization experiments were carried out using 8-9 week-old female BALB/c mice (Janvier, France) with 4 or 5 mice per experimental or control group.

Intramuscular Administration.

Each animal received 3 or 4 intramuscular injections at 3 week-intervals of 8 μg of naked or formulated DNA in a total volume of 100 μl (50 μl in each quadriceps). The injections was made using a 27×½ gauge needle fitted with a polyethylene tubing which limited the penetration to 2 mm.

Collection of Blood Samples.

Peripheral blood was collected by retro-orbital puncture 2 weeks after each injections.

Antibody-assays.

Serological responses were measured by enzyme-linked immunosorbant assay (ELISA). Maxisorb microtiter wells (Nunc, Denmark) were coated with 50 μl of recombinant β galactosidase protein (Roche Diagnostics, France) at 2 μg/ml in PBS for 1 night at 4° C. Wells were blocked with 3% BSA in PBS for 1 h and washed with 0.05% Tween-20 in PBS. Sera were diluted in PBS with 0.1% BSA and 0.05% Tween-20. A 50 μl-sample of serum per well was incubated for 2 h at 37° C. before washing and addition of horseradish peroxidase-conjugated goat anti-mouse IgG (Sigma, France). After 1 h-incubation and washing, 100 μl of O-phenylenediamine dihydrochloride (OPD) in phosphate-citrate buffer pH 5.0 and H₂O₂ were added as a substrate. Color development was stopped after 30 minutes with 50 μl of 1 N H₂SO₄ and the 490 nm absorbance measured. Antibody titers were calculated using the SOFTmax® PRO software (Molecular Devices) and expressed as the reciprocal of the final dilution which gave an absorbance equal to 0.2.

Assessment of Cellular Responses

Single cell suspensions were prepared from the spleens of mice 7 days after the third immunization. The spleen cells were treated with Tris-buffered NH₄Cl to lyse erythrocytes and resuspended at a concentration of 10×10⁶/ml in RPMI 1640 medium with Glutamax-I (Life Technologies) containing 10% FCS (v/v), 5×10⁻⁵ M 2-mercaptoethanol, 10 mM Hepes buffer, 1 mM sodium pyruvate and antibiotics (complete medium).

IFN-γ ELISPOT Assay

One million spleen cells in 100 μl complete medium were added to flat bottom Multiscreen 96-well plate (Millipore, France) coated with anti-IFN-γ rat antibody (Pharmingen, distributed by Becton Dickinson, France) and containing 100 μl of relevant or non relevant CTL peptide (2.5 μg/ml) for 24 hours at 37° C. under humidified atmosphere with 5% CO2. The positive control consisted in concanavaline A (1 μg/ml) stimulated cells. After washing with PBS-tween 20 0.05%, 100 μl of monoclonal biotin-conjugated rat antibody (Pharmingen) in PBS-tween 20 0.05% BSA 1% were added. After 1 hour incubation at 37° C., the plate was washed and 100 μl of extrAvidine-Alkaline Phosphatase conjugate (Sigma) were added. After another hour incubation, the plate was washed 3 times and 100 μl of Alkaline Phosphatase substrate solution (AP conjugated substrate kit, Biorad) were added. After 30 minutes, the revelation was stopped and spots counting was done using a binocular loupe.

Cytotoxic T-cell Assay

β-galactosidase directed specific lysis was assessed in a 4 hour ⁵¹Cr-release assay. Spleen cells were cultured in the presence of 0.1 μg/ml of specific CTL peptide in upright 75 cm² flask (Nunc) at a density of 10×10⁶ cells/ml in complete medium.. The synthetic peptides TPHPARIGL (T9L peptide) and IPQSLDSWWTSL (I12L) represent the naturally processed H-2L^{d}-restricted CTL epitope of β-galactosidase and HBsAg, respectively. The 2 peptides were synthesized by Neosystem, France. After 24 hours, 10 UI/ml IL-2 (Tebu, France) were added to the cultures and after a five-day incubation, the cells were recovered and assessed for CTL activity. Specific target cells for β-galactosidase CTL measurement were P815 pulsed with the T9L peptide. P815 cells pulsed with the I12L peptide were used as non-specific targets. In all cases, non specific lysis of P815 was less than 5% at 100:1 effector: target ratio.

II Results

Analysis of Antibody Response

As shown in FIG. 2, antibodies against β-galactosidase protein were detected in serum following 2 or 3 intramuscular administrations of DNA/cationized saccharide complexes. More importantly, the mice injected with DNA/glucidex-6 GTMA formulation showed higher specific antibody titers than mice injected with the same quantity of free DNA.

Analysis of the Cellular Response.

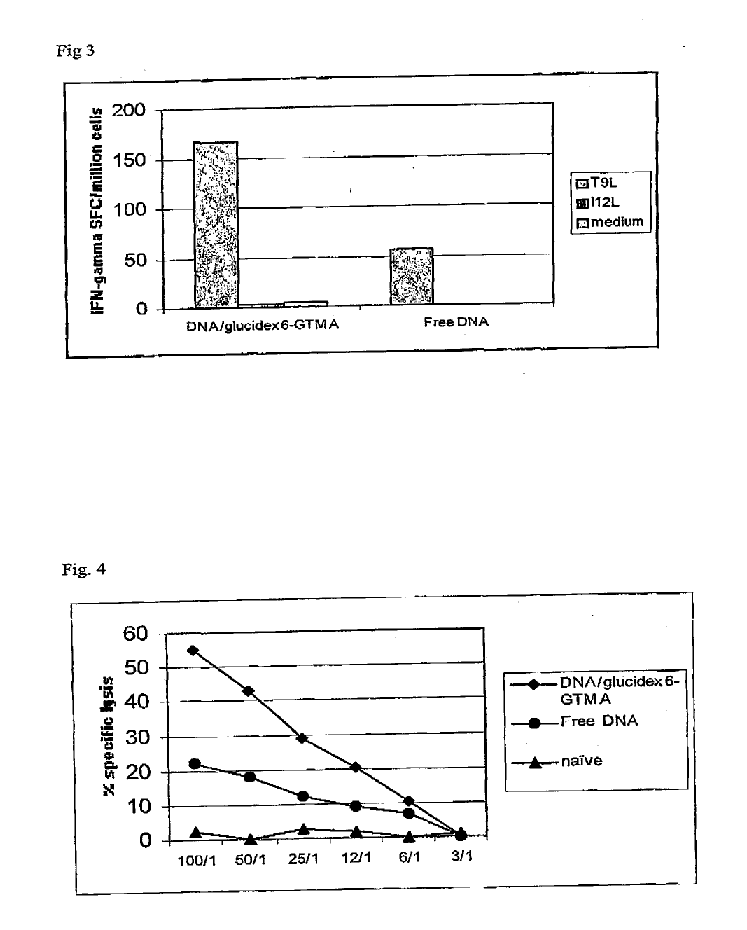

The ability of DNA/cationized saccharide complexes to induce a cellular immune response was first studied by IFN-γ ELISPOT assay using fresh spleen cells and the MHC-class I CTL peptide (T9L) specific of the β-galactosidase antigen (see Methods). As shown in FIG. 3, a significant number of spots, corresponding to IFN-γ secreting cells, was counted when freshly isolated spleen lymphocytes from mice immunized intramuscularly 3 times with DNA/cationized saccharide complexes were cultured 24 hours in the presence of T9L peptide. No spot was seen when spleen cells were incubated with medium alone or with a non relevant MHC-class I peptide (I12L), demonstrating the specificity of this secretion. The frequency was about 150 spots per million cells after 3 immunizations with DNA/glucidex-6 GTMA formulation, which was 3 times higher than the frequency obtained with free DNA.

The β galactosidase specific cellular response was also studied by a standard ⁵¹Cr release assay afer 5 days-in vitro stimulation with the β-galactosidase dominant MHC-class I peptide in bulk culture. A significant CTL activity was detected in mice which received 4 intramuscular administrations of 8 μg DNA/cationized saccharide complexes (FIG. 4). More importantly, the CTL activity after 4 immunizations with DNA/glucidex-6 GTMA formulation was higher than the activity obtained with free DNA.

We claim:

1. A particulate complex comprising a nucleic acid and a biodegradable cationized polyhydroxylated molecule, wherein said molecule has a charge up to approximately 1.0 meq/g.

2. A complex according to claim 1, wherein the nucleic acid is double or single stranded DNA or RNA, or a mixture thereof.

3. A complex according to claim 1, wherein the nucleic acid is a natural or chemically modified oligonucleotide or a derivative thereof.

4. A complex according to claim 1, wherein the nucleic acid is a natural or chemically modified polynucleotide or a derivative thereof.

5. A complex according to claim 1, wherein the biodegradable cationized polyhydroxylated molecule has a charge between approximately 0.1 and approximately 0.85 meq/g.

6. A complex according to claim 1, wherein the polyhydroxylated molecule is a saccharide comprising a cationic moiety.

7. A complex according to claim 6, wherein the saccharide is a polysaccharide

8. A complex according to claim 6, wherein the saccharide is an oligosaccharide.

9. A complex according to claim 6, wherein the saccharide is a monosaccharide.

10. A complex according to claim 6, wherein the cationic moiety comprises a secondary or tertiary amino group; quaternary ammonium ion; or a combination thereof.

11. A complex according to claim 10, wherein the quaternary ammonium ion is glycidyl trimethylammonium.

12. A complex according to claim 1, wherein the cationized polyhydroxylated molecule has a molecular weight of between approximately 0.18 kDa and approximately 1000 kDa.

13. A complex according to claim 12, wherein the cationized polyhydroxylated molecule has a molecular weight of between about 0.5 kDa and 500 kDa.

14. A complex according to claim 1, wherein the complex is of size between approximately 100 nm to approximately 10 μm.

15. A complex according to claim 1, wherein the complex has a charge ratio of cationized polyhydroxylated molecule to nucleic acid between approximately 0.3 to 1, and wherein the complex is globally negative.

16. A complex according to claim 1, wherein the complex has a charge ratio of cationized polyhydroxylated molecule to nucleic acid between 1 to approximately 20, and wherein the complex is globally positive.

17. A solution comprising a complex according to claim 16, wherein the solution further comprises excess cationized polyhydroxylated molecule that is not complexed to the nucleic acid.

18. A method for protecting a nucleic acid molecule when transfecting said molecule into a cell, said method comprising complexing the nucleic acid with a cationized polyhydroxylated molecule to form a particulate complex according to claim 1.

19. A method according to claim 18, wherein the complex has a charge ratio of cationized polyhydroxylated molecule to nucleic acid between approximately 0.3 and approximately 20.

20. A method for transfecting a nucleic acid molecule into a cell ex vivo, said method comprising complexing the nucleic acid with a cationized polyhydroxylated molecule to form a particulate complex according to claim 1, and transfecting the cell with the complex.

21. A method according to claim 20, wherein the complex has a charge ratio of cationized polyhydroxylated molecule to nucleic acid between approximately 0.3 and approximately 20.

22. A method for administering a nucleic acid molecule to a mammal, said method comprising complexing the nucleic acid with a cationized polyhydroxylated molecule to form a particulate complex according to claim 1, and administering the complex to the mammal.

23. A method according to claim 22, wherein the complex has a charge ratio of cationized polyhydroxylated molecule to nucleic acid between approximately 0.3 and approximately 20.

24. A method according to claim 22 wherein the administration of the complex is intramuscular.

25. A method according to claim 22, wherein the nucleic acid encodes an immunogenic antigen.

26. A method according to claim 22, wherein the nucleic acid encodes a therapeutic protein.

27. A pharmaceutical composition comprising the complex of claim 1.

28. A pharmaceutical composition according to claim 27 further comprising a transfection enhancer.

29. A pharmaceutical composition according to claim 28, wherein said transfection enhancer is selected from the group consisting of lipids, detergents, enzymes, peptides, and enzyme inhibitors.

30. A pharmaceutical composition according to claim 28, wherein said transfection enhancer comprises free cationized polyhydroxylated molecules not complexed to the nucleic acid.
